(19) United States
(12) Patent Application Publication
(10) Pub. No.: US 2008/0039381 A1
(43) Pub. Date: Feb. 14, 2008

(54) Lactalbumin for Inhibiting Angiogenesis

(76) Inventor: Catharina Svanborg, Lund (SE)

Correspondence Address: NIXON & VANDERHYE, PC, 901 NORTH GLEBE ROAD, 11TH FLOOR, ARLINGTON, VA, 22203, US

(21) Appl. No.: 10/590,938

(22) PCT Filed: Feb. 24, 2005
(86) PCT No.: PCT/GB05/00685

§ 371 Date: Jan. 9, 2007

(30) Foreign Application Priority Data
Feb. 26, 2004 (GB) 0404369.1
Feb. 26, 2004 (GB) 0404339.4
Feb. 26, 2004 (GB) 0404358.4

(51) Int. Cl. A61K 38/38; A61P 35/00

(52) U.S. Cl. 514/12

(57) ABSTRACT

The use of a biologically active complex of α-lactalbumin, selected from HAMLET (human α-lactalbumin made lethal to tumour cells) or a biologically active modification thereof, or a biologically active fragment of either of these, in the preparation of a medicament for use in the treatments of proliferative disease and in particular of mucosal cancers such as bladder cancer or malignant melanoma, as well as tumours of internal organs such as glioblastoma, as well as in the inhibition of angiogenesis.

The present invention relates to a method of treatment of humans, for conditions associated with unwanted cell or tissue proliferation, and to the use of biologically active complexes in the preparation of medicaments for the treatment of such conditions. In particular these conditions comprise malignant mucosal tumours or cancer such as bladder cancer, melanomas, cancers of internal organs, in particular, brain tumours, and any other condition, for instance cancers, where inhibition of angiogenesis is desirable.

Angiogenesis is the process of forming new blood vessels. It occurs normally in the human body at specific times in development and growth. For example, the embryo needs a vast network of arteries, veins, and capillaries. A process called vasculogenesis creates the primary network of vascular endothelial cells that will become major blood vessels. Later on, angiogenesis remodels this network into the small new blood vessels or capillaries that complete the child's circulatory system.

Proliferation of new blood vessels also takes place in adults. In women, angiogenesis is active a few days each month as new blood vessels form in the lining of the uterus during the menstrual cycle. Also, angiogenesis is necessary for the repair or regeneration of tissue during wound healing.

The vascular endothelial cell rarely divides, unless stimulated by angiogenesis. Angiogenesis is regulated by both activator and inhibitor molecules. Normally, the inhibitors predominate, blocking growth. Should a need for new blood vessels arise, angiogenesis activators increase in number and inhibitors decrease thus prompting the growth and division of vascular endothelial cells and, ultimately, the formation of new blood vessels.

Before the 1960s, cancer researchers believed that the blood supply reached tumors simply because pre-existing blood vessels dilated. But later experiments showed that angiogenesis is necessary for cancerous tumors to keep growing and spreading.

Tumor angiogenesis is the proliferation of a network of blood vessels that penetrates into cancerous growths, supplying nutrients and oxygen and removing waste products. It starts when tumor cells release molecules that send signals to surrounding normal host tissue, activating certain genes and proteins to encourage growth of new blood vessels.

Small activator molecules produced by the cancer cells signal angiogenesis in the surrounding tissue. More than a dozen different proteins, as well as several smaller molecules, have been identified as “angiogenic”. Among these are vascular endothelial growth factor (VEGF) and basic fibroblast growth factor (bFGF). VEGF and bFGF are produced by many kinds of cancer cells and by certain types of normal cells, too.

VEGF and bFGF are first synthesized inside tumor cells and then secreted into the surrounding tissue. The binding of either VEGF or bFGF to appropriate receptors activates a signalling cascade into the nucleus of the endothelial cells. The nuclear signal ultimately prompts a group of genes to make products needed for new endothelial cell growth.

The activation of endothelial cells by VEGF or bFGF sets in motion a series of steps toward the creation of new blood vessels. First, the activated endothelial cells produce matrix metalloproteinases (MMPs), a special class of degradative enzymes. These enzymes are then released from the endothelial cells into the surrounding tissue. The MMPs break down the extracellular matrix-support material that fills the spaces between cells and is made of proteins and polysaccharides.

Breakdown of this matrix permits the migration of endothelial cells. As they migrate into the surrounding tissues, activated endothelial cells begin to divide. Soon they organize into hollow tubes that evolve gradually into a mature network of blood vessels.

Although many tumors produce angiogenic molecules such as VEGF and bFGF, their presence is not enough to begin blood vessel growth. For angiogenesis to begin, these activator molecules must overcome a variety of angiogenesis inhibitors that normally restrain blood vessel growth.

Almost a dozen naturally occurring proteins can inhibit angiogenesis. Among this group of molecules, proteins called angiostatin, endostatin, and thrombospondin appear to be especially important. A finely tuned balance, between the concentration of angiogenesis inhibitors and of activators such as VEGF and bFGF, determines whether a tumor can induce the growth of new blood vessels. To trigger angiogenesis, the production of activators must increase as the production of inhibitors decreases.

The discovery of angiogenesis inhibitors raises the question of whether such molecules might therapeutically halt or restrain cancer's growth. Researchers have addressed this question in numerous experiments involving animals. In one striking study, mice with several different kinds of cancer were treated with injections of endostatin. After a few cycles of treatment, the initial (primary) tumor formed at the site of the injected cancer cells almost disappeared, and the animals did not develop resistance to the effects of endostatin after repeated usage.

The discovery that angiogenesis inhibitors such as endostatin can restrain the growth of primary tumors raises the possibility that such inhibitors might also be able to slow tumor metastasis.

It has been known for many years that cancer cells originating in a primary tumor can spread to another organ and form tiny, microscopic tumor masses (metastases) that can remain dormant for years. A likely explanation for this tumor dormancy is that no angiogenesis occurred, so the small tumor lacked the new blood vessels needed for continued growth.

One possible reason for tumor dormancy may be that some primary tumors secrete the inhibitor angiostatin into the bloodstream, which then circulates throughout the body and inhibits blood vessel growth at other sites. This could prevent microscopic metastases from growing into visible tumors.

Additional support for the idea that interfering with the process of angiogenesis can restrain tumor growth has come from genetic studies of mice. Scientists have recently created strains of mice that lack two genes, called Id1 and Id3, whose absence hinders angiogenesis. When mouse breast cancer cells are injected into such angiogenesis-deficient mutant mice, there is a small period of tumor growth, but the tumors regress completely after a few weeks, and the mice remain healthy with no signs of cancer. In contrast, normal mice injected with the same breast cancer cells die of cancer within a few weeks.

When lung cancer cells are injected into the same strain of angiogenesis-deficient mutant mice, the results are slightly different. The lung cancer cells do develop into tumors in the mutant, but the tumors grow more slowly than in normal mice and fail to spread (metastasize) to other organs. As a result, the mutant mice live much longer than normal mice injected with the same kinds of lung cancer cells.

As a result, it is now believed that inhibiting angiogenesis can slow down or prevent the growth and spread of cancer cells in humans, and as a result, a large number of angiogenesis inhibitors are currently being tested in cancer patients.

The inhibitors being tested fall into several different categories, depending on their mechanism of action. Some inhibit endothelial cells directly, while others inhibit the angiogenesis signaling cascade or block the ability of endothelial cells to break down the extracellular matrix.

HAMLET (human α-lactalbumin made lethal to tumour cells) (formerly known as MAL) is an active folding variant of alpha-lactalbumin (also represented as α-lactalbumin) that induces apoptosis in transformed cells but spares healthy differentiated cells (M. Svensson, et al., (2000) Proc Natl Acad Sci USA, 97, 4221-6). HAMLET has been shown to bind to the surface of tumour cells, to translocate into the cytoplasm and to accumulate in cell nuclei, where it causes DNA fragmentation (M. Svensson, et al., (2000) Proc Natl Acad Sci USA, 97, 4221-6). Biologically active complexes of this type, obtained from milk and particularly human milk, together with their use as antibacterial agents is described for example in EP-0776214.

The cellular targets for HAMLET have been examined by a combination of confocal microscopy and subcellular fractionation [Hakansson et al., 1999 Exp Cell Res. 246, 451-60]. HAMLET binds to the cell surface, and enters the cytoplasm where it interacts with and activates mitochondria. Finally, the protein enters the cell nuclei, where it accumulates.

The applicants have found that resistant and sensitive cells bind HAMLET to their surface with similar efficiency, suggesting that this is not the discriminating event. The nuclear accumulation, in contrast, occurs only in dying cells, suggesting that this step distinguishes sensitive tumour cells from resistant cells. By confocal microscopy, the nuclear accumulation appeared irreversible, suggesting the presence of nuclear targets that bind and retain HAMLET in the nuclear compartment.

To date, work reported with HAMLET has indicated that in-vitro, transformed cells are susceptible to HAMLET, which suggests that there it has an application in cancer therapy. The correlation with effects seen in vitro and those observed in vivo is not always straightforward however, in particular as conditions found in vivo vary depending upon the nature and position of the tumour. For instance, the different conditions found in different organs of the body can affect the stability and therefore the efficacy of any therapeutic reagent.

However, the applicants have found that HAMLET retains activity in vivo against human cells, and so it a useful anti-cancer therapy, in particular in certain instances.

Furthermore, it has now been found is that HAMLET also appears to have an inhibitory effect on angiogenesis, which is believed to be greater than would be expected simply from the tumour killing activity previously noted. This is unexpected in view of the highly selective nature of the cellular effects of the molecule. As a result, it increases the potential therapeutic range of the complex.

According to a first aspect of the present invention there is provided the use of a biologically active complex of α-lactalbumin, selected from HAMLET or a biologically active modification thereof, or a biologically active fragment of either of these, in the preparation of a medicament for use in the treatment of animals, in particular humans, for the proliferative disease, and/or to inhibit angiogenesis.

Particular examples of proliferative disease is cancer.

In one aspect, the invention provides a method of treating cancer in particular in humans, in-vivo, by applying to the tumour, HAMLET or a biologically active modification thereof, or a biologically active fragment of either of these. For this purpose, the biologically active complex is used in the preparation of a medicament for use in cancer therapy.

The applicants have found that HAMLET and complexes of this type produce unexpectedly good results when used in the treatment of mucosal tumours, particularly bladder cancer.

According to a second aspect of the present invention, there is provided the use of a biologically active complex of α-lactalbumin, selected from HAMLET or a biologically active modification thereof, or a biologically active fragment of either of these, in the preparation of a medicament for use in the treatment of human mucosal cancers.

The conditions found at mucosal surfaces can be quite unique in terms of properties such as p.H. and the like. Mucosal surfaces are found inter alia in the nasal passages, in the mouth, throat, oesophagus, lung, stomach, colon, vagina and bladder. Particular mucosal surfaces that may be treated with in accordance with the invention include throat, lung, colon and bladder surfaces which tumours. The invention is particularly applicable to the treatment of bladder cancer.

What has now been found however is that HAMLET also appears to have an inhibitory effect on angiogenesis, which is believed to be greater than would be expected simply from the tumour killing activity previously noted. This is unexpected in view of the highly selective nature of the cellular effects of the molecule. As a result, it increases the potential therapeutic range of the complex.

According to a fourth aspect of the present invention there is provided a biologically active complex of α-lactalbumin, selected from HAMLET or a biologically active modification thereof, or a biologically active fragment of either of these, in the preparation of a medicament for inhibiting angiogenesis.

Such medicaments can be used for treating cancers, and in particular solid cancers, and particularly rapidly proliferating solid tumors. In addition, however, it can be administered to slow tumour metastasis.

The mechanism by which HAMLET or a biologically active modification thereof achieves this result is not understood. It may be expected that some effects would be mediated by tumour cells. Specifically, as HAMLET kills tumour cells, the supply of angiogenesis activator molecules is reduced. However, the effects noted appear to indicate that additional effects are occurring. For instance, it seems possible that HAMLET has a direct effect on rapidly proliferating vascular cells.

It may also be used to treat other diseases where angiogenesis inhibition is desirable.

As a largely naturally occurring protein, it is believed that HAMLET will suffer from lower toxicity than entirely synthetic drugs. Furthermore, the immunogenicity of the complex is believed to be low.

The medicaments produced in accordance with the fourth aspect of invention are suitably pharmaceutical compositions in a form suitable for topical use, for example as creams, ointments, gels, or aqueous or oily solutions or suspensions. These may include the commonly known carriers, fillers and/or expedients, which are pharmaceutically acceptable.

Topical solutions or creams suitably contain an emulsifying agent for the protein complex together with a diluent or cream base.

The daily dose of the active compound varies and is dependant on the patient, the nature of the condition being treated etc. in accordance with normal clinical practice. As a general rule from 2 to 200 mg/dose of the biologically active complex is used for each administration.

In a further aspect of the invention, there is provided a method for inhibiting angiogenesis which comprises administering to a patient in need thereof, a biologically active complex of α-lactalbumin, selected from HAMLET or a biologically active modification thereof, or a biologically active fragment of either of these.

Preferred examples of the biologically active complex are illustrated above. Preferably the biologically active complex is administered in the form of a topical composition, also as described above.

As used herein, the term “HAMLET” refers to a biologically active complex of α-lactalbumin (which may or may not be human in origin), which is either obtainable by isolation from casein fractions of milk which have been precipitated at pH 4.6, by a combination of anion exchange and gel chromatography as described for example in EP-A-0776214, or by subjecting α-lactalbumin to ion exchange chromatography in the presence of a cofactor from human milk casein, characterized as C18:1 fatty acid as described in WO99/26979. Variants or derivatives of this complex with similar activity are described for example in International Patent Application No. PCT/IB03/01293.

The α-lactalbumin may be from various mammalian sources including human, bovine, sheep and goat milk, but is preferably human or bovine, and most preferably human. Recombinant forms of the protein may also be employed.

It has also been found that other reagents and specifically lipids such as oleic acid, are useful in the conversion of human α-lactalbumin to HAMLET. In particular, it has been reported previously that oleic acid (C18:1:9cis) is required for HAMLET production (M. Svensson, et al., (2000) Proc Natl Acad Sci USA, 97, 4221-6). More recently, it has been found that other fatty acids may act as co-factors in a similar way. Optimal cofactors for the conversion of α-lactalbumin to HAMLET are C18:1 fatty acids with a double bond in the cis conformation at position 9 or 11.

α-Lactalbumin is a 14.2 kDa globular protein with four α-helices (residues 1-34, 86-123) and an anti-parallel β-sheet (residues 38-82), linked by four disulphide bonds (61-77; 73-91; 28-111 and 6-120) (K. R. Acharya, et al., (1991) J Mol Biol, 221, 571-81). The native conformation of α-lactalbumin is defined by a high affinity Ca²⁺ binding site, co-ordinated by the side chain carboxylates of Asp82, Asp87 and Asp88, the carbonyl oxygens of Lys79 and Asp84, and two water molecules (K. R. Acharya, et al., (1991) J Mol Biol, 221, 571-81). The protein adopts the so called apo-conformation found in HAMLET when exposed to low pH, or in the presence of chelators, that release the strongly bound Ca²⁺ ion (D. A. Dolgikh, et al., (1981) FEBS Lett, 136, 311-5; K. Kuwajima, (1996) Faseb J, 10, 102-09).

In order to form biologically active complexes, α-lactalbumin generally requires both a conformational or folding change as well as the presence of a lipid cofactor. The conformational change is suitably effected by removing calcium ions from α-lactalbumin. In a preferred embodiment, this is suitably facilitated using a variant of α-lactalbumin which does not have a functional calcium binding site.

Biologically active complexes which contain such variants are encompassed by the term “modifications” of HAMLET as used herein. However, the applicants have found that, once formed, the presence of a functional calcium binding site, and/or the presence of calcium, does not affect stability or the biological activity of the complex. Biologically active complexes have been found to retain affinity for calcium, without loss of activity. Therefore complex of the invention may further comprise calcium ions.

Thus in particular, the invention uses a biologically active complex comprising alpha-lactalbumin or a variant of alpha-lactalbumin which is in the apo folding state, or a fragment of either of any of these, and a cofactor which stabilises the complex in a biologically active form, provided that any fragment of alpha-lactalbumin or a variant thereof comprises a region corresponding to the region of α-lactalbumin which forms the interface between the alpha and beta domains.

Suitably the cofactor is a cis C18:1:9 or C18:1:11 fatty acid or a different fatty acid with a similar configuration.

In a particular convenient embodiment, the biologically active complex used in the invention comprises

(i) a cis C18:1:9 or C18:1:11 fatty acid or a different fatty acid with a similar configuration; and

(ii) α-lactalbumin from which calcium ions have been removed, or a variant of α-lactalbumin from which calcium ions have been released or which does not have a functional calcium binding site; or a fragment of either of any of these, provided that any fragment comprises a region corresponding to the region of α-lactalbumin which forms the interface between the alpha and beta domains.

As used herein the expression “variant” refers to polypeptides or proteins which are homologous to the basic protein, which is suitably human or bovine α-lactalbumin, but which differ from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. Suitably variants will be at least 60% identical, preferably at least 70%, even more preferably 80% or 85% and, especially preferred are 90%, 95% or 98% or more identity.

When comparing amino acid sequences for the purposes of determining the degree of identity, programs such as BESTFIT and GAP (both from Wisconsin Genetics Computer Group (GCG) software package). BESTFIT, for example, compares two sequences and produces an optimal alignment of the most similar segments. GAP enables sequences to be aligned along their whole length and finds the optimal alignment by inserting spaces in either sequence as appropriate. Suitably, in the context of the present invention when discussing identity of sequences, the comparison is made by alignment of the sequences along their whole length.

The term “fragment thereof” refers to any portion of the given amino acid sequence which will form a complex with the similar activity to complexes including the complete α-lactalbumin amino acid sequence. Fragments may comprise more than one portion from within the full length protein, joined together. Portions will suitably comprise at least 5 and preferably at least 10 consecutive amino acids from the basic sequence.

Suitable fragments will be deletion mutants suitably comprise at least 20 amino acids, and more preferably at least 100 amino acids in length. They include small regions from the protein or combinations of these.

The region which forms the interface between the alpha and beta domains is, in human a-lactalbumin, defined by amino acids 34-38 and 82-86 in the structure. Thus suitable fragments will include these regions, and preferably the entire region from amino acid 34-86 of the native protein.

In a particularly preferred embodiment, the biologically active complex comprises a variant of α-lactalbumin in which the calcium binding site has been modified so that the affinity for calcium is reduced, or it is no longer functional.

It has been found that in bovine α-lactalbumin, the calcium binding site is coordinated by the residues K79, D82, D84, D87 and D88. Thus modification of this site or its equivalent in non-bovine α-lactalbumin, for example by removing one of more of the acidic residues, can reduce the affinity of the site for calcium, or eliminate the function completely and mutants of this type are a preferred aspect of the invention.

The Ca²⁺-binding site of bovine α-lactalbumin consists of a 3₁₀ helix and an α-helix with a short turn region separating the two helices (Acharya K. R., et al., (1991) J Mol Biol 221, 571-581). It is flanked by two disulfide bridges making this part of the molecule fairly inflexible. Five of the seven oxygen groups that co-ordinate the Ca²⁺ are contributed by the side chain carboxylates of Asp82, 87 and 88 or carbonyl oxygen's of Lys79 and Asp84. Two water molecules supply the remaining two oxygen's (Acharya K. R., et al., (1991) J Mol Biol 221, 571-581).

Site directed mutagenesis of the aspartic acid at position 87 to alanine (D87A) has previously been shown to inactivate the strong calcium-binding site (Anderson P. J., et al., (1997) Biochemistry 36, 11648-11654) and the mutant proteins adopted the apo-conformation.

Therefore in a particular embodiment, the aspartic acid residue at amino acid position 87 within the bovine α-lactalbumin protein sequence is mutated to a non-acidic residue, and in particular a non-polar or uncharged polar side chain.

Non-polar side chains include alanine, glycine, valine, leucine, isoleucine, proline, phenylalanine, methionine, tryptophan or cysteine. A particularly preferred examples is alanine. Uncharged polar side chains include asparagine, glutamine, serine, threonine or tyrosine.

In order to minimize the structural distortion in the mutant protein, D87 has also been replaced by an asparagine (N) (Permyakov S. E., et al., (2001) Proteins Eng 14, 785-789), which lacks the non-compensated negative charge of a carboxylate group, but has the same side chain volume and geometry. The mutant protein (D87N) was shown to bind calcium with low affinity (K-_{ca}2×10⁵M⁻¹) (Permyakov S. E., et al., (2001) Proteins Eng 14, 785-789). Such a mutant forms an element of the biologically active complex in a further preferred embodiment of the invention.

Thus particularly preferred variants for use in the complexes of the invention are D87A and D87N variants of α-lactalbumin, or fragments which include this mutation.

This region of the molecule differs between the bovine and the human proteins, in that one of the three basic amino acids (R70) is changed to S70 in bovine α-lactalbumin thus eliminating one co-ordinating side chain. It may be preferable therefore, that where the bovine α-lactalbumin is used in the complex of the invention, an S70R mutant is used.

The Ca²⁺ binding site is 100% conserved in a-lactalbumin from different species (Acharya K. R., et al., (1991) J Mol Biol 221, 571-581), illustrating the importance of this function for the protein. It is co-ordinated by five different amino acids and two water molecules. The side chain carboxylate of D87 together with D88 initially dock the calcium ion into the cation-binding region, and form internal hydrogen bonds that stabilise the structure (Anderson P. J., et al., (1997) Biochemistry 36, 11648-11654). A loss of either D87 or D88 has been shown to impair Ca2+ binding, and to render the molecule stable in the partially unfolded state (Anderson P. J., et al., (1997) Biochemistry 36, 11648-11654).

Further, mutant proteins with two different point mutations in the calcium-binding site of bovine α-lactalbumin may be used. For example, substitution of the aspartic acid at position 87 by an alanine (D87A) has been found to totally abolish calcium binding and disrupt the tertiary structure of the protein. Substitution of the aspartic acid by asparagine, the protein (D87N) still bound calcium but with lower affinity and showed a loss of tertiary structure, although not as pronounced as for the D87A mutant (Permyakov S. E., et al., (2001) Proteins Eng 14, 785-789). The mutant protein showed a minimal change in packing volume as both amino acids have the same average volume of 125 Å³, and the carboxylate side chain of asparagines allow the protein to co-ordinate calcium, but less efficiently (Permyakov S. E., et al., (2001) Proteins Eng 14, 785-789). Both mutant proteins were stable in the apo-conformation at physiologic temperatures but despite this conformational change they were biologically inactive. The results demonstrate that a conformational change to the apo-conformation alone is not sufficient to induce biological activity.

The structure of α-lactalbumin is known in the art, and the precise amino acid numbering of the residues referred to herein can be identified by reference to the structures shown for example in Anderson et al. supra. and Permyakov et al supra.

The medicaments produced in accordance with the second aspect of the invention are suitably pharmaceutical compositions in a form suitable for topical administration to the particular malignant mucosal tumour being treated. For instance, the composition may be in a form which is suitable for instillation into the bladder, where bladder cancer is the being treated. These may include the commonly known carriers, fillers and/or expedients, which are pharmaceutically acceptable. Suitably however, the composition instilled into the bladder will comprise a solution of the active agent in sterile water or saline.

Topical solutions or creams suitably contain an emulsifying agent for the protein complex together with a diluent or cream base may be more suitable for application to other malignant mucosal tumours. Such formulations can be applied directly to the tumour.

In addition, such topical compositions may be applied to treat malignant skin tumours, in particular melanoma. The applicants have found that HAMLET is particularly effective against melanoma cells. The use of these compositions in this way forms a further aspect of the invention.

The daily dose of the active compound varies and is dependant on the patient, the nature of the cancer being treated etc. in accordance with normal clinical practice. As a general rule from 200 mg to 1 g/dose of the biologically active complex is used for administration per day, preferably by intra-vesical instillation, over a period of at least 3 and preferably at least 5 days. In particular a dosage regime comprising 750 mg HAMLET per day for 5 days has proved beneficial.

The applicants have carried out studies on the effect of topical HAMLET treatment on bladder cancer. As reported below, the effects following intra-vesical instillation were extremely good.

In a further aspect of the invention, there is provided a method for treating mucosal cancers and in particular bladder cancer which comprises administering to a patient in need thereof, a biologically active complex of a-lactalbumin, selected from HAMLET or a biologically active modification thereof, or a biologically active fragment of either of these. The complex is suitably administered intra-vesically.

Preferred examples of the biologically active complex are illustrated above. Preferably the biologically active complex is administered in the form of a topical composition, also as described above.

The applicants have found that HAMLET and complexes of this type produce unexpectedly good results when infused directly into tumours of internal organs in vivo. In particular, it has been found that fluids found in the brain do not interfere with the activity.

According to a third aspect of the present invention, there is provided the use of a biologically active complex of α-lactalbumin, selected from HAMLET or a biologically active modification thereof, or a biologically active fragment of either of these, in the preparation of a medicament for infusion into tumours.

By infusing such a biologically active complex directly into tumours, it has been found that the size of tumours can be reduced, indicating that the effect of HAMLET in inducing apoptosis is occurring, in spite of the presence of body fluids which may include proteases. As a result, this treatment is particularly suitable for treatment of solid tumours of internal organs such as brain, liver, kidney, prostate and ovaries as well as in melanomas.

The selective nature of the effect of such complexes means that adjacent healthy tissue is unaffected, even if it comes into contact with the complex.

In particular, the invention is useful in the treatment of brain tumours, and also in toxin induced liver tumours. Fluids found in the brain in particular do not appear to interfere with the effects of Hamlet, to a surprising degree.

Malignant brain tumors represent a major therapeutic challenge in that no selective or efficient treatment is available. The majority of intra-cranial neoplasms originate from neuroglial cells, and form the heterogeneous group known as gliomas. They account for more than 60% of all primary brain tumors, and have a most unfavorable prognosis. Glioblastomas (GBMs) are the most malignant of the gliomas with a mean survival time of less than one year, and they constitute approximately one fourth of all intra-cranial tumors in neurosurgical and neuro-pathological series. In recent years, the surgical treatment of brain tumors has made significant technical advances. Microsurgery and neuro-navigation as well as new diagnostic high resolution imaging techniques have reduced morbidity, but the survival time has not improved. The GBMs remain inaccessible to complete surgical removal due to their invasive nature and diffuse infiltrating growth. As a consequence, the current treatment of these patients is palliative, involving partial tumor resection, radiotherapy and chemotherapy.

However, the applicants have found the biological complexes such as HAMLET provide a new tool in the treatment of in particular GBM. HAMLET killed GBM tumor cells by an apoptosis-like mechanism in vitro, and the effect was selective, as healthy cells were spared. Furthermore, HAMLET maintained these properties in vivo, in the human GBM xeno-graft model. Regional infusion of HAMLET into established human GBM tumors significantly delayed tumor development and the onset of pressure symptoms. HAMLET killed the tumor cells by an apoptosis-like mechanism also in vivo, as shown by the TUNEL assay and by histopathology. There was no evidence of necrosis and the effect was selective, as no histo-pathological changes were detected in the surrounding intact brain. In vitro treatment of biopsy spheroids confirmed the efficient killing of malignant cells by HAMLET, as compared to benign meningiomas. The results thus suggest that HAMLET can be used to treat GBM.

The medicaments produced in accordance with the third aspect of the invention are suitably pharmaceutical compositions in a form suitable for intra-tumoral administration to the particular solid tumour being treated. For instance, the composition may be in a form which is suitable for infusion into a tumour. These may include the commonly known carriers, fillers and/or expedients, which are pharmaceutically acceptable. Suitably however, the composition for infusion will comprise a solution of the active agent in a saline solution.

The dose of the active compound varies and is dependant on the patient, the nature of the cancer being treated etc. in accordance with normal clinical practice. As a general rule from 2 mg to 200 mg/dose of the biologically active complex is infused into the tumour at any one time.

The study reported herein investigated the therapeutic efficacy of HAMLET in the human GBM xeno-graft model. It shows that HAMLET maintains the ability to selectively induce apoptosis-like death in GBMs in vivo, in spite of the contact with brain fluid. It was found that intra-tumoral administration of HAMLET prolongs survival in rats with human glioblastomas (GBMs) by selective induction of tumor cell apoptosis. Invasively growing human GBMs were established in nude rats by xeno-transplantation of human biopsy spheroids, and the therapeutic effect of HAMLET was compared to a-lactalbumin; the native, folded variant of the same protein.

Intra-cerebral, convection enhanced delivery of HAMLET dramatically reduced the intra-cranial tumor volume and delayed the onset of pressure symptoms in the tumor bearing rats. HAMLET failed to induce apoptosis in healthy brain tissue adjacent to the tumor and did not cause toxic side effects after infusion of therapeutic concentrations into the brains of healthy rats. The results identify HAMLET as a potential new tool in cancer therapy, and in particular to the control of GBM progression.

The results also show that there was a marked difference in disease progression between the xeno-transplanted rats receiving HAMLET and α-lactalbumin (p<0.001). This illustrates how differences in biological activity can arise from a change in protein fold, and from the association with specific cofactors like oleic acid.

In a further aspect of the invention, there is provided a method for treating cancer which comprises infusing into a tumour or into the area thereof, a biologically active complex of α-lactalbumin, selected from HAMLET or a biologically active modification thereof, or a biologically active fragment of either of these.

In particular, the complex is suitably administered using suitable infusion equipment, and in particular convection enhanced delivery techniques (CED) have been found to be particularly effective.

Preferred examples of the biologically active complex are illustrated above.

According to a fourth aspect of the present invention there is provided a biologically active complex of α-lactalbumin, selected from HAMLET or a biologically active modification thereof, or a biologically active fragment of either of these, in the preparation of a medicament for inhibiting angiogenesis.

Such medicaments can be used for treating cancers, and in particular solid cancers, and particularly rapidly proliferating solid tumors. In addition, however, it can be administered to slow tumour metastasis.

The mechanism by which HAMLET or a biologically active modification thereof achieves this result is not understood. It may be expected that some effects would be mediated by tumour cells. Specifically, as HAMLET kills tumour cells, the supply of angiogenesis activator molecules is reduced. However, the effects noted appear to indicate that additional effects are occurring. For instance, it seems possible that HAMLET has a direct effect on rapidly proliferating vascular cells.

It may also be used to treat other diseases where angiogenesis inhibition is desirable.

As a largely naturally occurring protein, it is believed that HAMLET will suffer from lower toxicity than entirely synthetic drugs. Furthermore, the immunogenicity of the complex is believed to be low.

The medicaments produced in accordance with the fourth aspect invention are suitably pharmaceutical compositions in a form suitable for topical use, for example as creams, ointments, gels, or aqueous or oily solutions or suspensions. These may include the commonly known carriers, fillers and/or expedients, which are pharmaceutically acceptable.

Topical solutions or creams suitably contain an emulsifying agent for the protein complex together with a diluent or cream base.

The daily dose of the active compound varies and is dependant on the patient, the nature of the condition being treated etc. in accordance with normal clinical practice. As a general rule from 2 to 200 mg/dose of the biologically active complex is used for each administration.

In a further aspect of the invention, there is provided a method for inhibiting angiogenesis which comprises administering to a patient in need thereof, a biologically active complex of α-lactalbumin, selected from HAMLET or a biologically active modification thereof, or a biologically active fragment of either of these.

Preferred examples of the biologically active complex are illustrated above. Preferably the biologically active complex is administered in the form of a topical composition, also as described above.

The invention will now be particularly described by way of example with reference to the accompanying Figures in which:

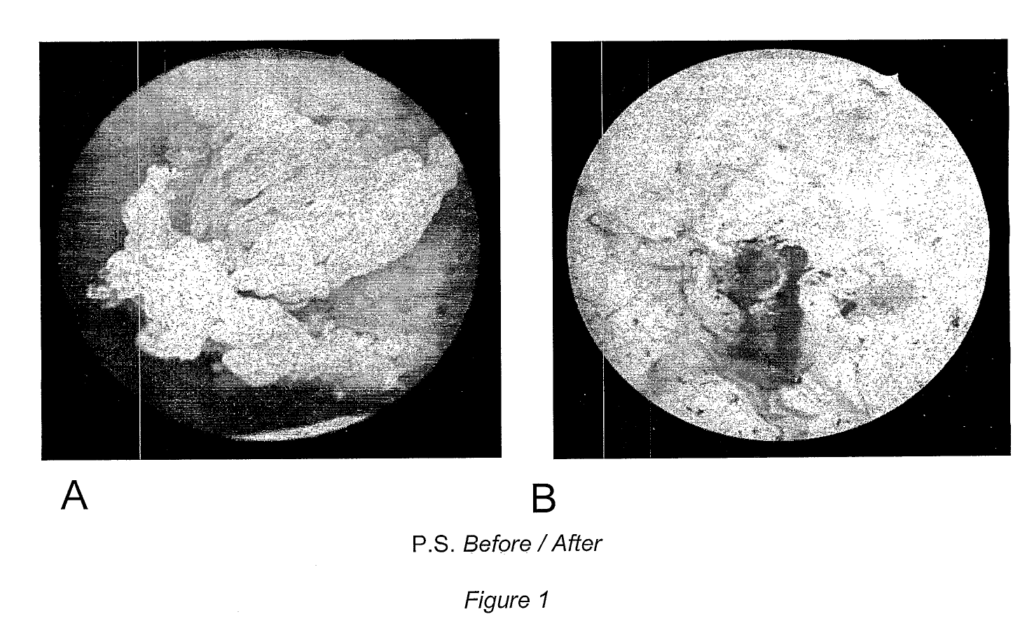

FIG. 1 shows an endoluminal photograph of a bladder cancer, taken before and after treatment in accordance with the invention;

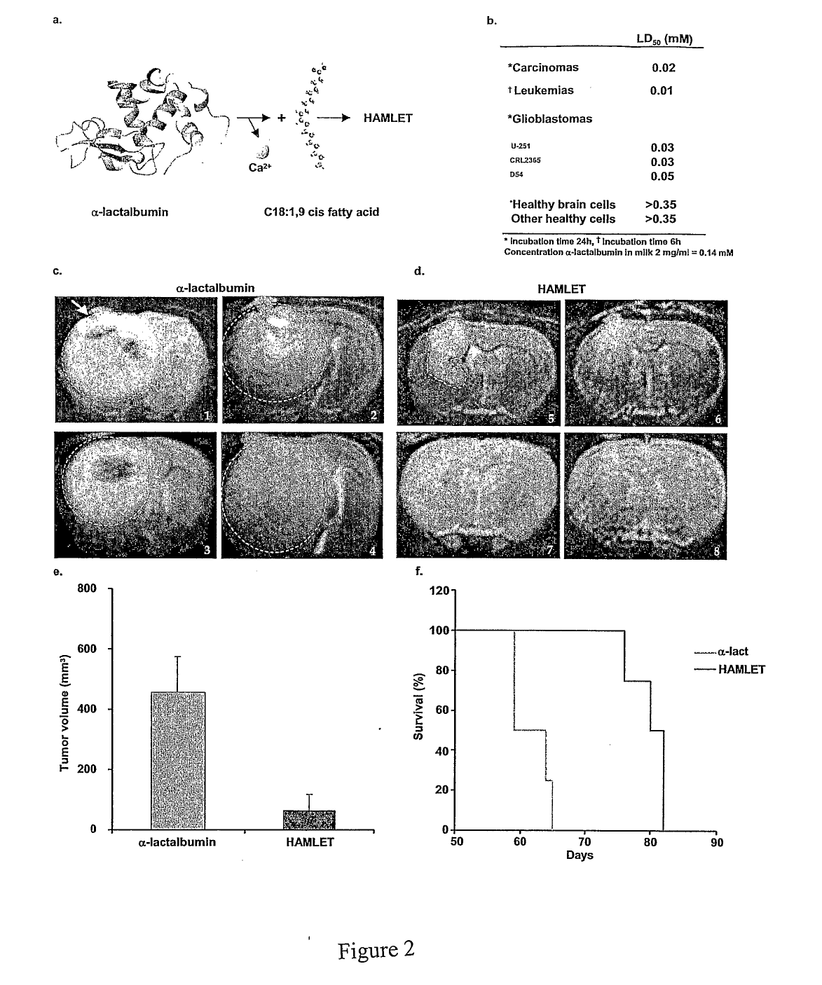

FIG. 2. HAMLET—structure and function in vitro and in vivo in human GEM tumour xeno-grants. (a) HAMLET is formed from native α-lactalbumin by removal of Ca²⁺ and by addition of the C18:1, 9 cis fatty acid. The figure is based on the α-lactalbumin crystal structure. (b) Sensitivity of GBM cell lines to HAMLET. LD₅₀=concentration required to kill 50% of the cells in 6/24 hours. (c and d) Human GBM tumour spheroids (injected at the arrow) were allowed to establish for one week prior to a 24-hour infusion with HAMLET (n=10) or α-lactalbumin (n=10). MRI scans of individual tumours in rats treated with α-lactalbumin (1-4) or HAMLET (5-8) were performed two months post infusion. (e) The mean tumour size was significantly smaller in the HAMLET-infused animals than in the α-lactalbumin treated group (p<0.01). (f) Symptoms of elevated intra-cranial pressure were recorded and occurred after about two months in the α-lactalbumin controls, but the onset of pressure symptoms was delayed in rats receiving HAMLET (p<0.001).

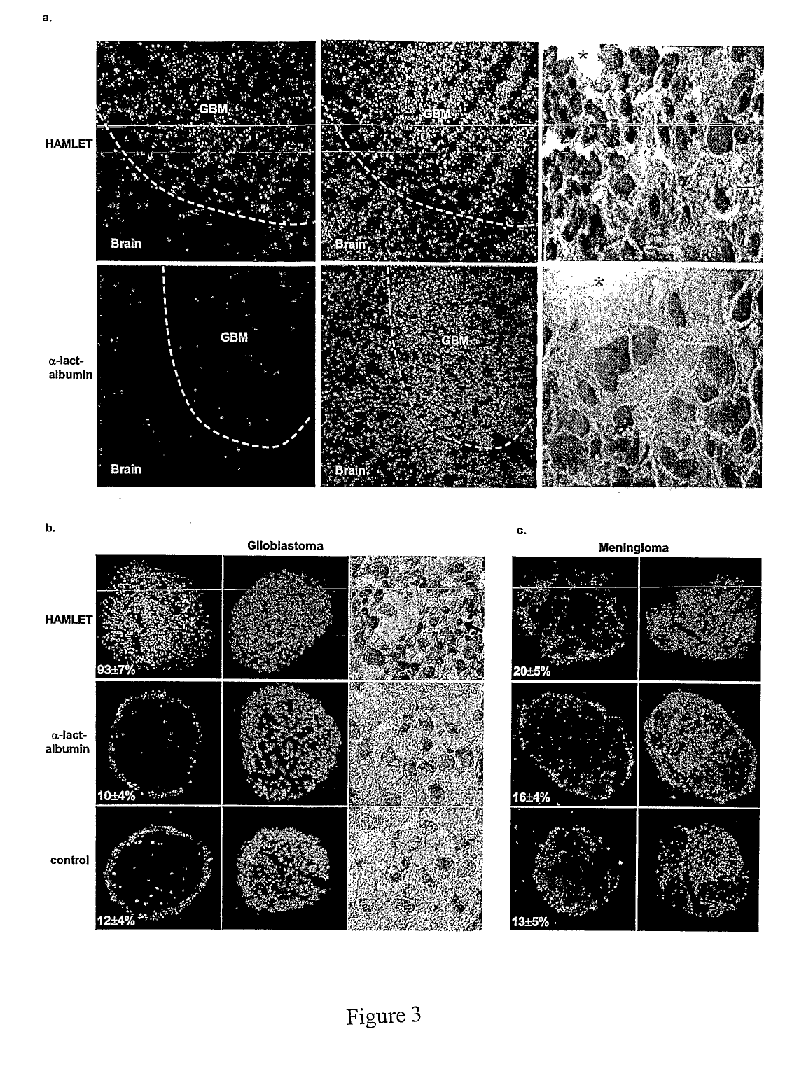

FIG. 3. Apoptosis induction by HAMLET. (a) Brain tissue sections were obtained from tumour bearing rats, twelve hours after CED of HAMLET or α-lactalbumin. HAMLET caused abundant apoptosis within the tumour area, as shown by TUNEL staining, (green fluorescence, left panels) and pycontic apoptotic tumour cell nuclei (right panels, magnification 600×). No apoptosis was observed in healthy brain tissue surrounding the tumour in the HAMLET treated animals or in the a-lactalbumin treated group. Cell nuclei were visualized using Propidium iodide staining of cellular DNA (red fluorescence). (b) GBM spheroids were treated with HAMLET or α-lactalbumin in vitro and apoptosis-induction was examined. HAMLET induced apoptosis (green fluorescence) was seen throughout the human GBM spheroids but not in spheroids derived from benign meningiomas (c). α-lactalbumin did not stimulate apoptosis in either the GBM or meningioma spheroids (magnification 360×). Hyper-chromatic and pycnotic apoptotic cells (arrow in b) were found in the HAMLET-treated spheroids but not in the α-lactalbumin group (magnification 450×).

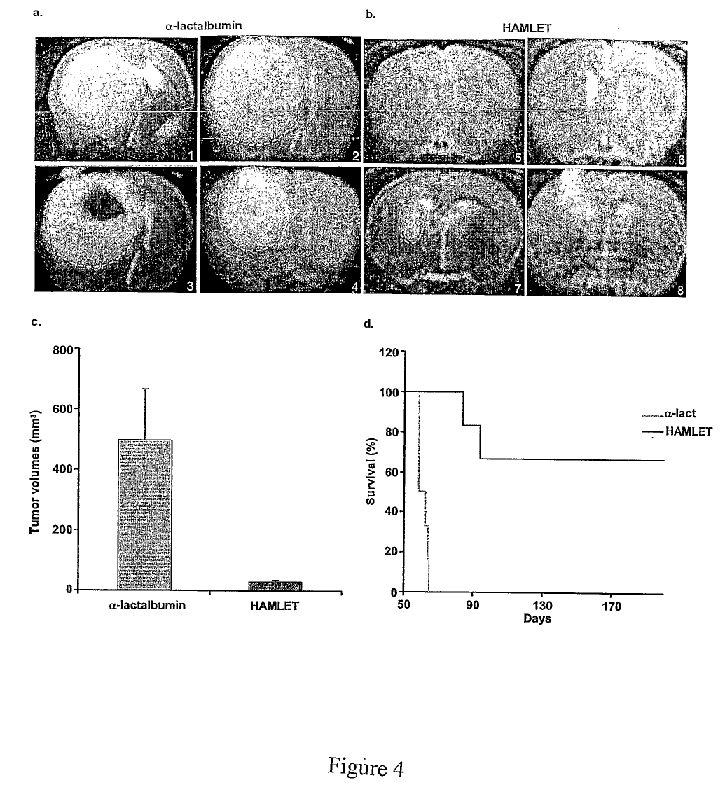

FIG. 4. Xeno-transplantation of GBM spheroids following pre-treatment with HAMLET or α-lactalbumin. Six animals in each group were xeno-transplanted with established human GBM spheroids (4-5 in each group) which had been pre-treated for three hours with HAMLET or α-lactalbumin. All rats receiving α-lactalbumin pre-treated GBM cells, developed large tumours (a, 1-4). Four out of six animals that received HAMLET treated spheroids showed no signs of tumour development and survived for at least 210 days (b, 5-8). The two rats in the HAMLET group that developed tumours at all showed significantly smaller tumours(c, p<0.01), and the onset of pressure symptoms was delayed (d, p<0.01).

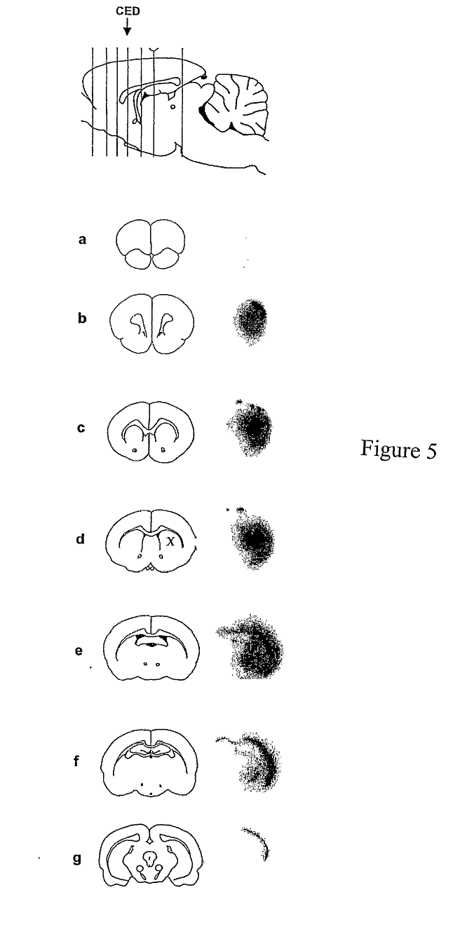

FIG. 5. Distribution of radio-labelled HAMLET. (2-10×10⁶ PPM) after infusion into brains of healthy rats (n=3, magnification 90×). The letters indicate the position of the sections and x the infusion site. (a) frontal lobe, (b) basal ganglia, (c) thalamus and (g) substantia nigra.

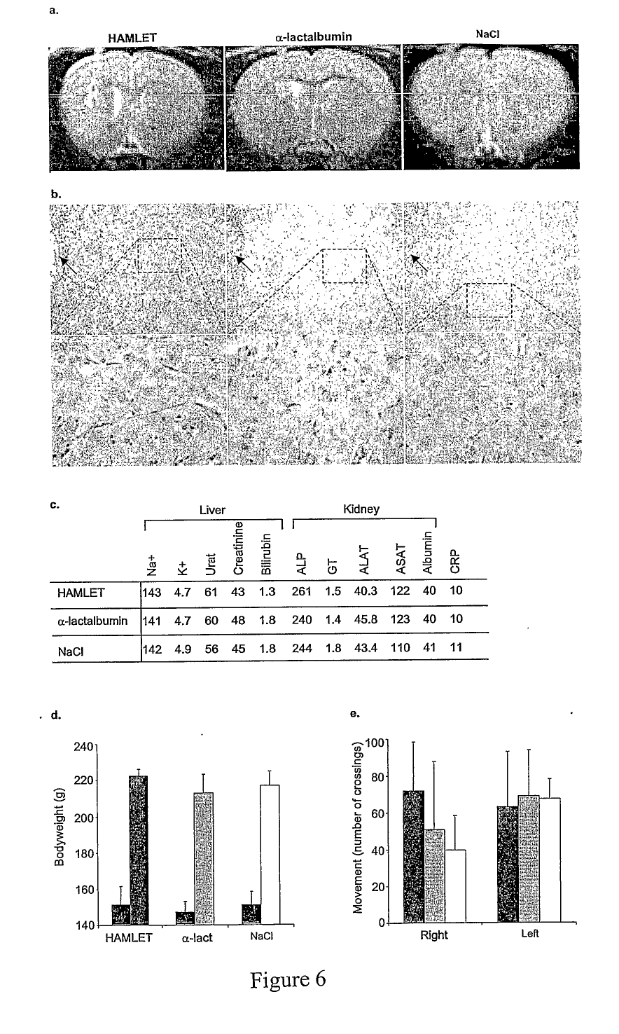

FIG. 6. Evaluation of toxicity. Healthy rats were treated with 0.7 mM of HAMLET, α-lactalbumin or 0.15 M of NaCl (n=5 in each group). Potential toxicity was analysed three weeks post infusion. (a) T2-weighted signals in MR images show small cystic lesions at the infusion site but no radiological signs of toxicity. (b) Histopathology in serial brain sections from the infused hemisphere showed no evidence of toxicity in healthy brains but some tissue destruction adjacent to the infusion site (arrow, htx-eosin, magnification 100× and 400×). (c) Biochemical markers of liver and kidney function revealed no significant toxic effects (p>0.05 in both groups). (d) The body weight increase did not differ between the groups (p>0.5). Hatched bars show body weight values before infusion and filled bars are the values three weeks post infusion (e) Open field test of movement was not affected (p>0.05, dark grey=HAMLET, light grey=α-lactalbumin, white=NaCl).

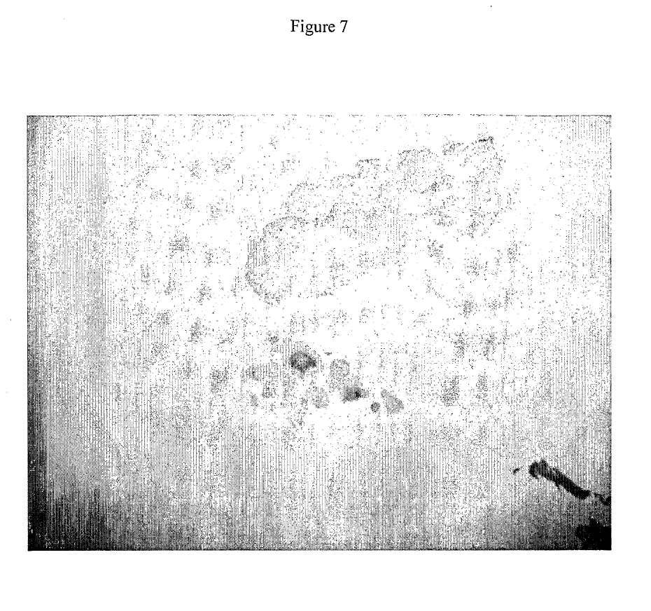

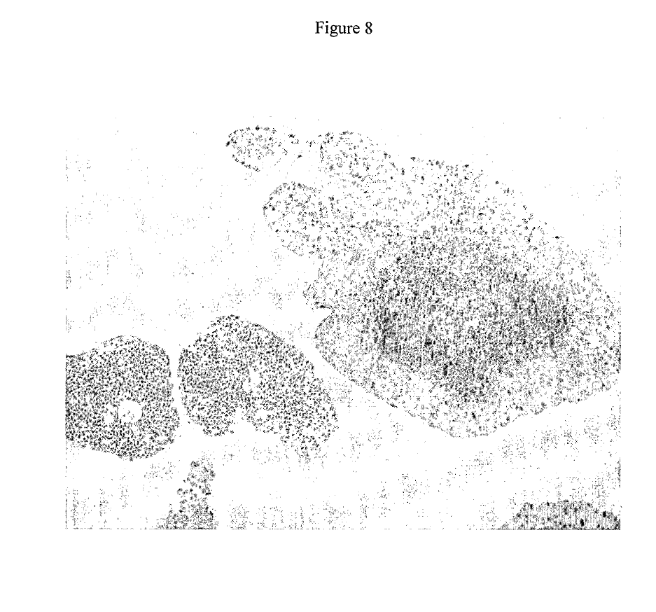

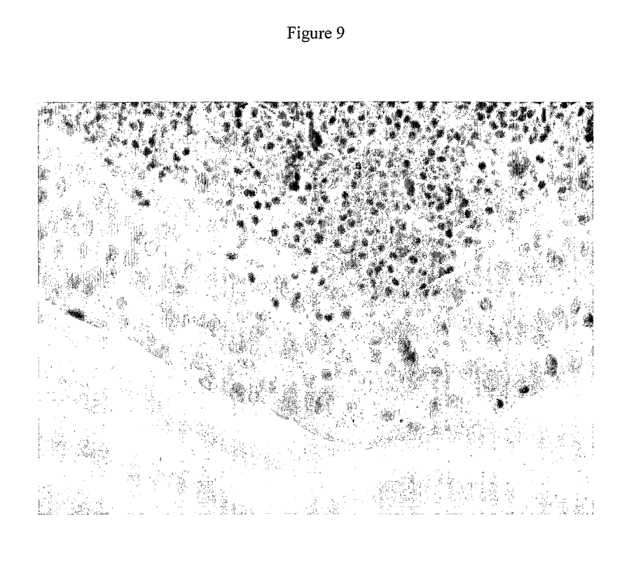

FIGS. 7 to 10 show a progressively enlarged tissue section illustrating the disassembly of blood vessels in a human bladder papilloma after topical HAMLET treatment for 5 days.

### EXAMPLE 1

Intra-Vesical Instillation of Hamlet in Patients with Cancer of the Urinary Bladder

Preparation of Substance and Randomisation of Patients

Donors of breastmilk were non-smokers and were screened for HIV prior to preparation of HAMLET. Alpha-lactalbumin was purified from human milk whey by ammonium sulphate precipitation followed by phenyl-Sepharose chromatography and size-exclusion chromatography. Excess milk from the hospital milk bank was used according to regulations for administration to premature babies. HAMLET was generated from native α-lactalbumin on an oleic acid conditioned ion-exchange chromatography column, as described in the literature. The eluted fractions were dialysed against distilled water, lyophilised and stored at −20° C.

Furthermore, HAMLET was screened for bacterial contamination and was stored as dry substance in −20° C.

Study Design:

Patients awaiting surgery for a newly diagnosed, or recurrent uro-epithelial cancer of the urinary bladder, were invited to participate in the study. After informed consent the patients were subjected to cystoscopy to assess the tumour size and to document the lesion with endoluminal photography. After treatment and prior to surgery, cystoscopy was repeated to re-assess tumour size and endoluminal photography was carried out.

Intra-vesical instillation of HAMLET was performed in the out-patient clinic under close surveillance. The instillations were given once daily, and repeated for five days. After urethral catheterisation the bladder was completely emptied and the urine was collected for analysis. HAMLET (25 mg/ml, 30 ml) was deposited in the bladder, the catheter removed, and the patients were asked to too keep the instillation for at least for two hours. To decrease the diuresis the patients were asked to avoid fluid intake for four hours before, and immediately after the instillation. Urine samples were provided prior to, and from the first voided urine after each instillation.

The HAMLET instillations were scheduled without interrupting or delaying the routine handling of the patients.

Patients: Seven male patients were included in the study. Based on standard tumour classification, the patients were assigned to three groups, A, B and C.

Patients A1 and A2 (92 and 86 years old) had poorly differentiated muscle invasive uro-epithelial cancer of the urinary bladder (T4g3). Due to their high age, these patients had been subjected to palliative measurements, such as repeated trans-urethral resections of the tumour (T-TUR), analgetics, and clinical observation. Both patients suffered mild lower urinary tract symptoms with frequency and urge, but they were otherwise healthy despite their high ages.

Patients B1, B2, B3, and B4 (37, 75, 70, 82 years old respectively) had superficial papillomatous bladder tumours (TAg1-2). Patients B1 and B3 had newly diagnosed tumours, and patients B2 and B4 had recurrencies of previously known highly differentiated, superficial bladder tumours (TAg1). Patients B1 and B4 were healthy except for their tumour, while patient B2 suffered high blood pressure in combination with cardio-sclerosis. Patient B3 had high blood pressure and chronic bronchitis. Patient C1 (72 years) had previously known multi-focal manifestations of cancer in situ (CIS) of the urinary bladder. He had been subjected to intra-vesical instillations of Bacille Calmette Guerin (BCG) one year prior to inclusion. Bladder biopsies had initially shown response to the BCG treatment. Prior to inclusion recurrence of CIS had been diagnosed in biopsy specimens. This patient was otherwise healthy.

Treatment Outcome:

All patients were given daily intra-vesical instillations of HAMLET on five consecutive days. None of the patients experienced any side effects of the instillations, and there were no reaction in systemic inflammatory parameters like CRP, fever or peripheral neutrophil counts.

Patients A1 and A2 had difficulties keeping the HAMLET solution in the bladder, due to lower urinary tract dysfunctions. The therapeutic effect could not be evaluated. Patient B1 showed a nearly complete reduction of the tumour after the five daily HAMLET instillations.

Patient B2 showed a small reduction in tumour size, but a marked change in the tumour character. Prior to treatment the tumour was brittle and bled on contact, but after treatment, the surface was “dry”. Patient B3 carried a papillomatous tumour on the left bladder wall that was too big to be captured in one photograph. After the intravesical HAMLET instillations, the ocular tumour size assessment showed a reduction in size of 50%. Patient B 4, had two small exophytic tumours on the left bladder neck. There was no apparent reduction in tumour size, but a marked change in tumour character with surface atrophy.

Patient C1 was difficult to evaluate due to the absence of exophytic tumour growth. Prior to the HAMLET instillations 3/3 bladder biopsies (“mapping”) showed cancer in situ, but after the 5 day treatment, only ⅓ biopsies was positive.

TUNEL positive, apoptotic cancer cells were detected.

Biopsies from macroscopically healthy bladder mucosa were taken from five of the patients. There was no effect of the HAMLET treatment identified in these biopsies.

We conclude that HAMLET treatment induces apoptosis in bladder cancer cells and significanly influences the volume and macroscopic appearance of the tumour.

### EXAMPLE 2

Preparation of HAMLET

HAMLET was produced from apo α-lactalbumin by ion exchange chromatography, on a DEAE-trisacryl M (BioSepra, France) column preconditioned with the C18:1, 9 cis fatty acid (Svensson et al. Proc. Natl. Acad. Sci USA, 97:4221-4226). ¹²⁵I labeling of HAMLET (1 mg/ml) was by the lactoperoxidase method (Hakansson et al., Proc Natl. Sci USA 92:8064-8068).

Tumour Tissues and Cell Lines

Tumour biopsies were collected, with the approval of the Medical Ethics Committee at the Haukeland University Hospital (Bergen, Norway), from a GBM of the right frontal lobe and a parasagittal meningioma. Spheroids with a diameter of 300 μm were cultured and used for transplantation (Bjerkvig et al., J. Neurosurg 72:463-475). Gliomal cell lines were: ATCC CRL 2365, D-54MG and U-251MG. The A549 lung carcinoma line was ATCC CCL 185. A single cell suspension of fully differentiated murine brain cells was prepared by dissection of the brain from a full-grown mouse, dissociation in DMEM medium (GibcofBRL, Life Technologies Ltd. Paisley, Scotland) with 1% trypsin (Sigma Chemicals Inc., St. Louis, Mo., USA) for 30 minutes at room temperature, addition of 0.24% DNase and 1% FCS (Sigma Chemicals Inc., St. Louis, Mo., USA) followed by mechanical disruption. The viability was >99%.

Xeno-Transplantation of Human GBMs to Nude Rats

All experiments were approved by The National Animal Research Authority and conducted according to The European Convention for the Protection of Vertebrates Used for Scientific purposes. Nude rats (Han:rnu/rnu Rowett) bred at the Haukeland hospital, were anaesthetized by intra-peritoneal injection of Equitisin, placed in a stereo-tactic frame (David Kopf, model 900, Tujunga, Calif., USA) for trepanation, and about 5-10 μl of PBS containing 5 biopsy spheroids was injected into the striatum. The rats were monitored daily until they developed symptoms of increased intra-cranial pressure such as passivity, clumsiness and paresis. The tumor mass was quantified by magnetic resonance scans, using a 1.5 Tesla Siemens Magnetom Vision instrument (Erlangen, Germany) and with a finger-coil for cerebral analysis. The mean time from transplantation of about 1 million cells to pressure symptoms was about two months, at which time the animals were sacrificed.

Convection Enhanced Delivery of HAMLET to the Intact Brain

HAMLET or α-lactalbumin (0.7 mM in 0.15 M NaCl) was administered through a 26 Gauge cannula connected to an osmotic mini pump (ADO, Alzet Inc., Mountainview, Calif., USA). The region of the tumor was infused at 8 μl/hour over 24 hours before the cannula was removed. ¹²⁵I radio-labeled HAMLET (0.7 mM in 0.15 M NaCl, 2-10×10⁶ PPM) was administered as described. The distribution of HAMLET was verified by autoradiography on serial brain sections from the entire infused hemisphere.

Tissue Analysis

Brains were rapidly embedded in Tissue-Tec (Sakura Finetek Inc., Torrance, Calif., USA) and frozen in liquid nitrogen. Serial axial 10 μm sections were cut on a Reichert Jung Cryostat (Reichert, Vienna, Austria). Apoptotic cells were detected by the TUNEL assay (Roche, Basel, Switzerland), and cover-slipped with a mounting medium (Vectashield, Vector Labs Inc., Burlingame, Calif., USA). Cell nuclei were counter-stained with Propidium iodide (10 μg/ml, for 30 seconds) and examined in a Leica scanner. Parallel sections were stained with Hematoxylin-Eosin and mounted in Entellan (Merck, Darmstadt, Germany). Sections without freezing artifacts and with an acceptable signal/noise ratio for FITC (TUNEL) and TRITC (Propidium iodide) were identified, and one representative section from the center of each tumor or spheroid was subjected to morphometric analysis. FITC and TRTTC positive nuclear profiles were clearly visible above background and were counted from printed pictures. Results are expressed as TUNEL positive in per cent of Propidium iodide positive nuclei.

In Vitro Treatment with HAMLET

Established spheroids (4-5 in each group) were moved to serum free medium, incubated for three hours with HAMLET or a-lactalbumin, and immediately transplanted into the brains of nude rats. For analysis of apoptosis, spheroids were transferred back to DMEM, incubated for another 21 hours and examined after serial sectioning by the TUNEL assay with morphometry. The cell lines were cultured as described (Hakansson et al. supra.), detached, harvested, washed and exposed to HAMLET or a-lactalbumin for 24 hours. Apoptosis was determined as the loss of cell viability assessed by Trypan blue exclusion (% dead cells per 100 counted cells) and DNA fragmentation was detected by electrophoresis (Zhivotovsky et al. FEBS Lett. 351:150-154).

Toxicity Tests

Rats receiving HAMLET (0.7 mM), α-lactalbumin (0.7 mM) or NaCl (0.15 M) were analyzed three weeks post infusion. The tumor mass was quantified by magnetic resonance scans, using a 1.5 Tesla Siemens Magnetom Vision instrument (Erlangen, Germany) and with a finger-coil for cerebral analysis. Histopathology was determined as described above using Hematoxylin-Eosin. Biochemical markers of liver and kidney function and CRP were quantified. The body weight was recorded before infusion as well as three weeks post infusion. Brain function was assessed by the open field test. Rats were placed in an open field box (100×100 cm) surrounded by black walls (20 cm). The floor was divided into 25 identical sectors (20×20 cm) by white stripes. The animals were placed in the central sector and their movements were scored manually for six minutes. Each motility count represented the crossing of a sector border with both hind limbs and the direction was noted as right or left. The experiments were performed between 10 am and 2 pm in a soundproof room, in a blinded manner.

Statistical Analysis

Groups were compared with T test, one-way-ANOVA (post hoc LSD), and survival by Kaplan-Meier-analysis.

Results

The GBM Xeno-Transplant Model

Two experimental models have been developed to study GBM treatment in vivo. Gliomal cell lines grow efficiently in vitro, and invariably produce intra-cerebral tumors after transplantation; but these tumors are not invasive in vivo, and thus less suitable as a model of the human disease. Human GBM biopsy spheroids, in contrast, maintain their invasive growth behavior after xeno-transplantation into nude rats. The in vitro spheroid culture step is essential to obtain a reproducible tumor mass, and to synchronize the appearance of clinical symptoms. This model thus offers a relevant treatment model of human GBM disease, and may be combined with CED of therapeutic molecules into the tumor area.

HAMLET Inhibits the Growth of Human Gliomal Xeno-Grafts

Experimental GBMs were established by xeno-transplantation of human GBM biopsy spheroids into the nude rat brain (Engebraaten et al. J. Neurosurg. 90:125-132). The xeno-grafts showed the infiltrative growth characteristics of human GBM (FIG. 2c), and the control rats developed symptoms after about two months (FIG. 2f).

CED was used to administer HAMLET (0.7 mM) into the xeno-grafted area of the brain. Native, folded a-lactalbumin was used as a control. Prior to treatment, the tumor cells were allowed one week to become integrated into the host brain.

HAMLET or a-lactalbumin were then administered by CED for 24 hours. Two animals in each group died during anesthesia, and four animals in each group were sacrificed twelve hours later. Their brains were immediately frozen for histology, TUNEL assay, and morphometric analysis.

The remaining animals were monitored daily for two months, and tumor volumes were assessed by MRI after seven weeks when the α-lactalbumin treated control animals developed symptoms. Large GBM-transplants with high T2-weighted signals could be observed in all the α-lactalbumin treated animals, with a mean tumor volume of 456 (range 292-485) mm³ (FIGS. 2c and e). The HAMLET-infused rats showed significantly smaller tumor volumes (FIGS. 2d and e, mean 63, range 10-131 mm³, p<0.01). HAMLET treatment also delayed the onset of pressure symptoms. Rats receiving α-lactalbumin developed symptoms on day 59, and by day 65, all animals had been sacrificed. At this time, all animals in the HAMLET-treated group remained asymptomatic (FIG. 2f, p<0.01). The HAMLET treated rats eventually developed pressure symptoms and died with typical GBM tumors, showing polymorphic cell types and pseudo-pallisading on histological examination.

Selective Tumor Cell Apoptosis in Human GBM Xeno-Grafts

Apoptosis induction was examined in vivo using the TUNEL assay, which labels DNA-strand breaks. Morphometric analysis on tissues obtained twelve hours after completion of CED showed that 33±7% of the HAMLET-treated GBM cells were TUNEL-positive compared to 2±2% in the a-lactalbumin group (FIG. 3a, p<0.001). The apoptotic effect was confirmed by routine histopathology, which showed typical pycnotic and condensed nuclei in the HAMLET treated animals (FIG. 3a). The host brain surrounding the tumor showed no evidence of apoptosis or necrosis after CED of HAMLET or α-lactalbumin into the transplanted hemisphere (FIG. 3a).

HAMLET Induces Apoptosis-Like Death in GBM Biopsy Spheroids In Vitro

The ability of HAMLET to induce apoptosis in the GBM cells was verified in vitro. Biopsy spheroids from the same human GBM were exposed in vitro to HAMLET and apoptotic cells were identified by the TUNEL-assay, with Propidium iodide counter-staining to visualize the total cell population. HAMLET-treated GBM spheroids showed abundant TUNEL-staining throughout the entire volume of the spheroids (FIG. 3b). By morphometry, 93±7% (mean±SD) of the nuclei were found to be apoptotic. TUNEL-positive cells were observed throughout the entire volume of the GBM spheroids at concentrations of 0.35 mM or higher, confirming the relevance of the concentration selected for the therapeutic studies. By histopathology, pycnotic and condensed nuclei were observed in the HAMLET exposed GBM (see arrow in FIG. 3b).

Control GBM spheroids treated with α-lactalbumin were seen to shed a few apoptotic cells from the surface, but no TUNEL positive cells were seen in the interior of the spheroids, and there was no difference in the frequency of apoptotic cells between the GBM spheroids exposed to α-lactalbumin and the medium control. Both were significantly different from the HAMLET treated spheroids (p<0.001). HAMLET did not trigger apoptosis in biopsy spheroids from a patient with a benign meningioma (FIG. 3c).

In Vitro Pre-Treatment of GBM Spheroids Confirmed the Therapeutic Effect

GBM biopsy spheroids were exposed to HAMLET in vitro for three hours and then xenotransplanted into nude rat brains, as described. Spheroids treated with α-lactalbumin served as controls. The tumor size was estimated by MRI scans after two months. Tumors developed in all rats that received α-lactalbumin treated spheroids (FIG. 4a). The mean tumor size was 496 (range 286-696) mm³, and the rats developed symptoms from day 56 (FIGS. 4c and d). At this time, the HAMLET treated spheroids had detectable tumors and these tumors were smaller than in the α-lactalbumin controls with a mean volume of 31 (range 28-34) mm³ (FIGS. 4b and c). The rats with smaller tumors developed pressure symptoms after 84 days. The remaining animals were tumor free and asymptomatic at the time of sacrifice, 210 days after transplantation (FIG. 4d, p<0.01).

HAMLET Reaches Throughout the Infused Hemisphere

The efficiency of HAMLET administration by CED was investigated. ¹²⁵I radio-labeled HAMLET (2-10×10⁶ PPM was infused by CED with the needle inserted in the striatum and the distribution of HAMLET throughout the brain was detected by auto-radiography on serial brain sections (FIG. 5). HAMLET was shown to reach the entire infused hemisphere from the forebrain to the mesencephalon, twelve hours after completion of the CED.

Therapeutic Concentrations of HAMLET are not Toxic for Healthy Brain Tissue

Potential brain toxicity of HAMLET was examined by MRI and histopathology three weeks after CED into the striatum of healthy rats. In analogy with previous experiments, α-lactalbumin or saline served as controls. By MRI, small cystic lesions were seen at the infusion site, but there were no signs of edema or tissue damage in the surrounding brain, including the cortex which had been penetrated by the infusion cannula (see T2 weighted scans in FIG. 6a). There were no radiological differences between the HAMLET and the control groups.

Histopathological analyses of the infused brains showed some tissue destruction adjacent to the infusion site, with increased cellularity comprising reactive microglia, macrophages and a few reactive astrocytes. There were no significant neuro-pathological signs of toxicity in the surrounding brain parenchyma and no differences between the HAMLET treated and the control groups (FIG. 6b).

Biochemical markers and body weight changes were monitored three weeks after infusion. No differences were observed between the HAMLET, α-lactalbumin and NaCl treated rats (p>0.05 in all groups) (FIGS. 6c and d).

Changes in movement and behavior were assessed by the open field test three weeks after infusion. The rats were placed in an open field checkerboard and the number of crossings to a new square was recorded. No significant movement disorders were detected (FIG. 6e).

### EXAMPLE 3

Effects of Intra-Vesical Instillation of HAMLET on Blood Supply to Tumours in Patients with Cancer of the Urinary Bladder

Following the trial reported in Example 1 above, A biopsy sample of a treated tumour was taken at the end of this treatment, and the results are shown in FIGS. 7 to 10. As is clear from these figures, the endothelial lining is missing and blood corpuscles are present throughout the core of the tumor, indicating that angiogenesis has been inhibited.

1-21. (canceled)

22. A method for treating humans for proliferative disease, and/or to inhibit angiogenesis which comprises administering to said a patient, a biologically active complex of a-lactalbumin, selected from HAMLET or a biologically active modification thereof, or a biologically active fragment of either of these.

23. A method for treating a mucosal tumour which comprises administering to said tumour in a patient in need thereof, a biologically active complex of α-lactalbumin, selected from HAMLET or a biologically active modification thereof, or a biologically active fragment of either of these.

24. A method according to claim 23 wherein the mucosal tumour is bladder cancer.

25. A method according to claim 24 wherein the biologically active complex is administered by intra-vesical instillation.

26. A method according to claim 25 wherein from 200 mg to 1 g of the biologically active complex is administered in a single dosage unit.

27. A method according to claim 26 wherein the dosage unit is repeated on at least 5 days.

28. A method according to claim 27 wherein the dosage is given on consecutive days.

29. A method for treating cancer which comprises infusing into a tumour, a biologically active complex of α-lactalbumin, selected from HAMLET or a biologically active modification thereof, or a biologically active fragment of either of these.

30. A method according to claim 29 wherein the complex is administered in the form of a composition further comprising a saline carrier.

31. A method according to claim 29 wherein the complex is infused using convection enhanced delivery (CED).

32. A method according to claim 29 wherein the tumour is a tumour of the brain, liver, kidney, prostate and ovaries.

33. A method according to claim 32 wherein the tumour is a brain tumour.

34. A method according to claim 33 wherein the tumour is human glioblastoma.

35. The method of claim 22 wherein said method comprises inhibiting angiogenesis which comprises administering to a patient in need thereof, a biologically active complex of α-lactalbumin, selected from HAMLET or a biologically active modification thereof, or a biologically active fragment of either of these.

36. (canceled)

37. A method for treating malignant melanoma, which method comprises applying a biologically active complex of α-lactalbumin, selected from HAMLET or a biologically active modification thereof, or a biologically active fragment of either of these, to the melanoma.

38. The method of claim 29 wherein said method comprises treating cancer in particular in humans, in-vivo, by applying to the tumour, a biologically active complex comprising HAMLET or a biologically active modification thereof, or a biologically active fragment of either of these.

39. (canceled)
